(19) United States
(12) Patent Application Publication
(10) Pub. No.: US 2017/0259078 A1
(43) Pub. Date: Sep. 14, 2017

(54) IMPLANTABLE OPTICAL STIMULATION LEADS AND METHODS OF MAKING AND USING

(71) Applicant: Boston Scientific Neuromodulation Corporation, Valencia, CA (US)

(72) Inventor: Joshua Dale Howard, Sacramento, CA (US)

(21) Appl. No.: 15/450,969

(22) Filed: Mar. 6, 2017

Related U.S. Application Data
(60) Provisional application No. 62/305,342, filed on Mar. 8, 2016.

(51) Int. Cl. A61N 5/06

(52) U.S. Cl. CPC A61N 5/0601; A61N 2005/063; A61N 2005/067

(57) ABSTRACT

An optical stimulation lead including a lead body including a distal portion and a proximal portion; an optical fiber disposed within the lead body to emit light from a distal portion of the optical fiber; and at least one flex zone disposed along the distal portion of the lead body. Each flex zone includes an elastomeric structure that is made of a flexible material different from the lead body. Optical stimulation leads can alternatively or additionally include filters or stabilizers. In addition, optical stimulation leads can utilize light sources, such as laser diodes, LEDs, or OLEDs, instead of optical fibers.

### CROSS-REFERENCE TO RELATED APPLICATIONS

This application claims the benefit under 35 U.S.C. §119(e) of U.S. Provisional Patent Application Ser. No. 62/305,342, filed Mar. 8, 2016, which is incorporated herein by reference.

### FIELD

The present invention is directed to the area of implantable optical stimulation systems and methods of making and using the systems. The present invention is also directed to implantable optical stimulation leads having elements that facilitate implantation or selective stimulation, as well as methods of making and using the leads and optical stimulation systems.

### BACKGROUND

Implantable optical stimulation systems can provide therapeutic benefits in a variety of diseases and disorders. For example, optical stimulation can be applied to the brain either externally or using an implanted stimulation lead to provide, for example, deep brain stimulation, to treat a variety of diseases or disorders. Optical stimulation may also be combined with electrical stimulation.

Stimulators have been developed to provide therapy for a variety of treatments. A stimulator can include a control module (for generating light or electrical signals sent to light sources in a lead), one or more leads, and one or more light sources coupled to, or disposed within, each lead. The lead is positioned near the nerves, muscles, or other tissue to be stimulated.

### BRIEF SUMMARY

One embodiment is an optical stimulation lead including a lead body including a distal portion and a proximal portion; an optical fiber disposed within the lead body to emit light from a distal portion of the optical fiber; and at least one flex zone disposed along the distal portion of the lead body. Each flex zone includes an elastomeric structure that is made of a flexible material different from the lead body.

In at least some embodiments, the elastomeric structure includes a rubber material. In at least some embodiments, the lead body includes a cladding of the optical fiber. In at least some embodiments, the elastomeric structure includes features on an exterior surface of the elastomeric structure. In at least some embodiments, the features are pyramidal features, bumps, hemispherical features, ridged features, or any combination thereof on the exterior surface.

In at least some embodiments, the lead further includes a cushion region between the elastomeric structure and the optical fiber, where the cushion region has a radial width of at least 0.03 millimeters. In at least some embodiments, the elastomeric structure extends around an entire circumference of the optical fiber.

Another embodiment is an optical stimulation system that includes any of the optical stimulation leads described above; and a control module coupleable to the optical stimulation lead, the control module including a housing and a light source disposed in the housing and configured and arranged to provide light to the optical fiber of the optical stimulation lead.

Yet another embodiment is an optical stimulation lead including a lead body including a distal portion and a proximal portion; at least one light emitter disposed within the lead body to emit light from a distal portion of the lead body; and light filters disposed along the distal end of the lead to receive the emitted light from the at least one light emitter and permit passage of light through the light filter of a predetermined wavelength or range of wavelengths. At least two of the light filters permit passage of light for different wavelengths or different range of wavelengths from each other.

In at least some embodiments, the at least one light emitter includes at least one optical fiber. In at least some embodiments, the at least one optical fiber includes at least one multimode optical fiber.

In at least some embodiments, the at least one light emitter includes at least one light source selected from the group consisting of a light emitting diode, an organic light emitting diode, a laser diode, or any combination thereof. In at least some embodiments, the at least one light source is a plurality of light sources, wherein at least two of the light sources produce light of different wavelengths from each other.

In at least some embodiments, the light filters are configured and arranged to be turned on or off by received electrical signals.

A further embodiment is an optical stimulation lead including a lead body including a distal portion and a proximal portion; at least one light emitter disposed within the lead body to emit light from a distal portion of the lead body; and stabilizers disposed along the distal portion of the lead body. Each stabilizer includes a body disposed around the lead body, at least one tab extending from the body, and at least one suture hole formed in the at least one tab.

In at least some embodiments, the at least one stabilizer is molded with the lead body to form a unitary arrangement of the lead body and the at least one stabilizer. In at least some embodiments, the at least one stabilizer is affixed to the lead body by overmolding the at least one stabilizer over the lead body. In at least some embodiments, the at least one stabilizer is slideably disposed on the lead body.

Yet another embodiment is an optical stimulation system including any of the optical stimulation leads describe above; and a control module coupleable to the optical stimulation lead, the control module including a housing, and an electronic subassembly disposed in the housing to provide electrical signals or light to the at least one light emitter of the optical stimulation lead.

### BRIEF DESCRIPTION OF THE DRAWINGS

Non-limiting and non-exhaustive embodiments of the present invention are described with reference to the following drawings. In the drawings, like reference numerals refer to like parts throughout the various figures unless otherwise specified.

For a better understanding of the present invention, reference will be made to the following Detailed Description, which is to be read in association with the accompanying drawings, wherein:

FIG. 1 is a schematic side view of one embodiment of an optical stimulation system that includes a lead coupled to a control module, according to the invention;

FIG. 2A is a schematic side view of one embodiment of the control module of FIG. 1 configured and arranged to couple to an elongated device, according to the invention;

FIG. 2B is a schematic side view of one embodiment of a lead extension configured and arranged to couple the elongated device of FIG. 2A to the control module of FIG. 1, according to the invention;

FIG. 3 is a schematic cross-sectional view of one embodiment of an optical stimulation lead with at least one flex zone, according to the invention;

FIG. 4 is a schematic side view of one embodiment of an optical stimulation lead with filters, according to the invention;

FIG. 5 is a schematic side view of one embodiment of an optical stimulation lead with stabilizers, according to the invention;

FIG. 6 is a schematic overview of one embodiment of components of a stimulation system, including an electronic subassembly disposed within a control module, according to the invention.

### DETAILED DESCRIPTION

The present invention is directed to the area of implantable optical stimulation systems and methods of making and using the systems. The present invention is also directed to implantable optical stimulation leads having elements that facilitate implantation or selective stimulation, as well as methods of making and using the leads and optical stimulation systems.

In some embodiments, the implantable optical stimulation system only provides optical stimulation. In other embodiments, the stimulation system can include both optical and electrical stimulation. In at least some of these embodiments, the optical stimulation system can be a modification of an electrical stimulation system to also provide optical stimulation. Suitable implantable electrical stimulation systems that can be modified to also provide optical stimulation include, but are not limited to, a least one lead with one or more electrodes disposed along a distal end of the lead and one or more terminals disposed along the one or more proximal ends of the lead. Leads include, for example, percutaneous leads, paddle leads, and cuff leads. Examples of electrical stimulation systems with leads are found in, for example, U.S. Pat. Nos. 6,181,969; 6,516,227; 6,609,029; 6,609,032; 6,741,892; 7,244,150; 7,450,997; 7,672,734; 7,761,165; 7,783,359; 7,792,590; 7,809,446; 7,949,395; 7,974,706; 6,175,710; 6,224,450; 6,271,094; 6,295,944; 6,364,278; and 6,391,985; U.S. Patent Applications Publication Nos. 2007/0150036; 2009/0187222; 2009/0276021; 2010/0076535; 2010/0268298; 2011/0004267; 2011/0078900; 2011/0130817; 2011/0130818; 2011/0238129; 2011/0313500; 2012/0016378; 2012/0046710; 2012/0071949; 2012/0165911; 2012/0197375; 2012/0203316; 2012/0203320; 2012/0203321; 2012/0316615; and 2013/0105071; and U.S. patent application Ser. Nos. 12/177,823 and 13/750,725, all of which are incorporated by reference in their entireties.

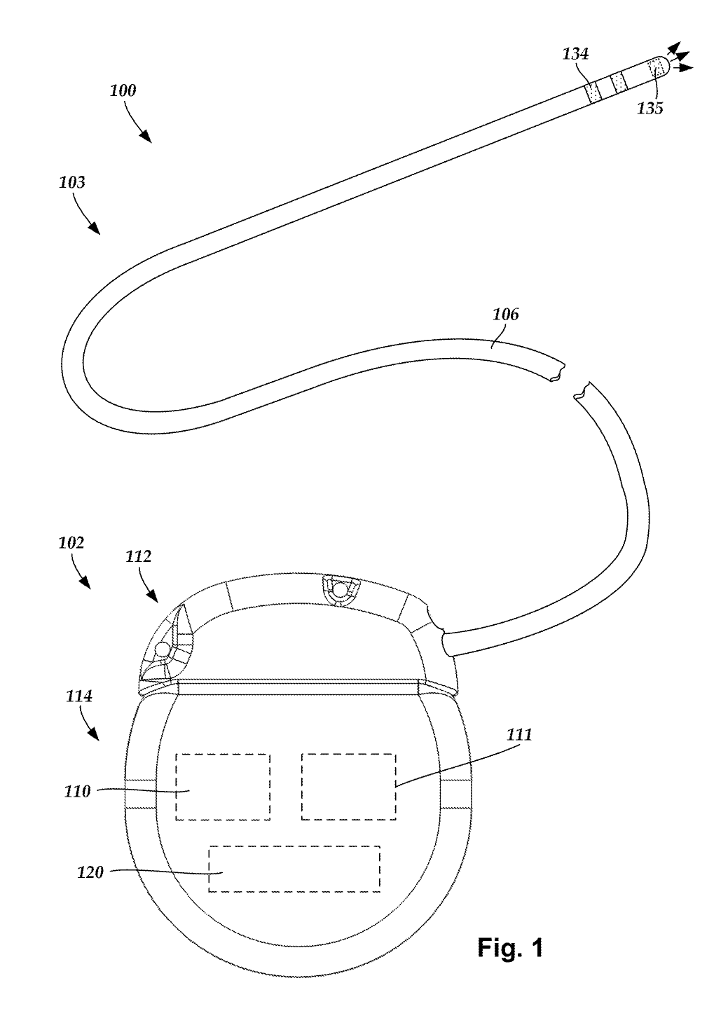

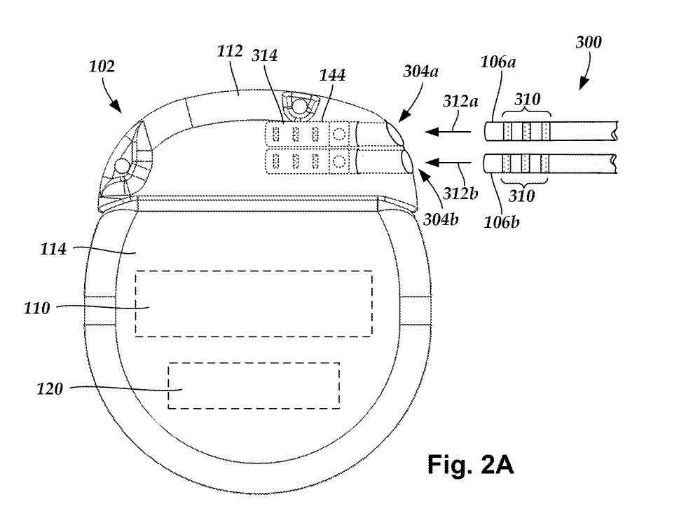

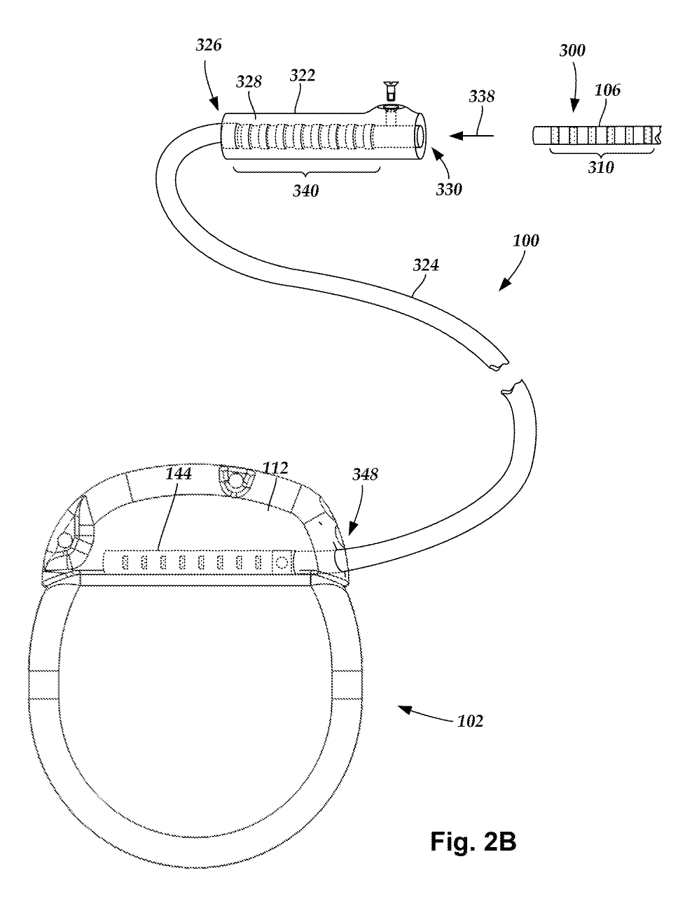

FIG. 1 illustrates schematically one embodiment of an optical stimulation system 100. The optical stimulation system includes a control module (e.g., a stimulator) 102 and a lead 103 coupleable to the control module 102. The lead 103 includes one or more lead bodies 106. In FIG. 1, the lead 103 is shown having a single lead body 106. In FIG. 2B, the lead 103 includes two lead bodies. It will be understood that the lead 103 can include any suitable number of lead bodies including, for example, one, two, three, four, five, six, seven, eight or more lead bodies 106.

At least one light emitter 135 is provided at a distal end of the lead 103. The light emitter 135 can be a light source, such as a light emitting diode (LED), laser diode, organic light emitting diode (OLED), or the like, or can be a terminus of a light transmission element, such as an optical fiber, in which case the light source is distant from the distal end of the light (for example, in the control module or in a proximal portion of the lead). Optionally, the lead can also include one or more electrodes 134 disposed along the lead body 106, and one or more terminals (e.g., 310 in FIG. 2A-2B) disposed along each of the one or more lead bodies 106 and coupled to the optional electrodes 134 by conductors (not shown). In at least some embodiments, one or more terminals (e.g., 310 in FIG. 2A-2B) may also be used to convey electrical signals to a light source that acts as the light emitter 135 by conductors (not shown) extending along the lead.

The lead 103 can be coupled to the control module 102 in any suitable manner. In some embodiments, the lead is permanently attached to the control module 102. In other embodiments, the lead can be coupled to the control module 102 by a connector (e.g., connector 144 of FIG. 2A). In FIG. 2A, the lead 103 is shown coupling directly to the control module 102 through the connector 144. In at least some other embodiments, the lead 103 couples to the control module 102 via one or more intermediate devices, as illustrated in FIG. 2B. For example, in at least some embodiments one or more lead extensions 324 (see e.g., FIG. 2B) can be disposed between the lead 103 and the control module 102 to extend the distance between the lead 103 and the control module 102. Other intermediate devices may be used in addition to, or in lieu of, one or more lead extensions including, for example, a splitter, an adaptor, or the like or combinations thereof. It will be understood that, in the case where the stimulation system 100 includes multiple elongated devices disposed between the lead 103 and the control module 102, the intermediate devices may be configured into any suitable arrangement.

The control module 102 can include, for example, a connector housing 112 and a sealed electronics housing 114. An electronic subassembly 110 and an optional power source 120 are disposed in the electronics housing 114. A control module connector 144 is disposed in the connector housing 112. The control module connector 144 is configured and arranged to make an electrical connection between the lead 103 and the electronic subassembly 110 of the control module 102.

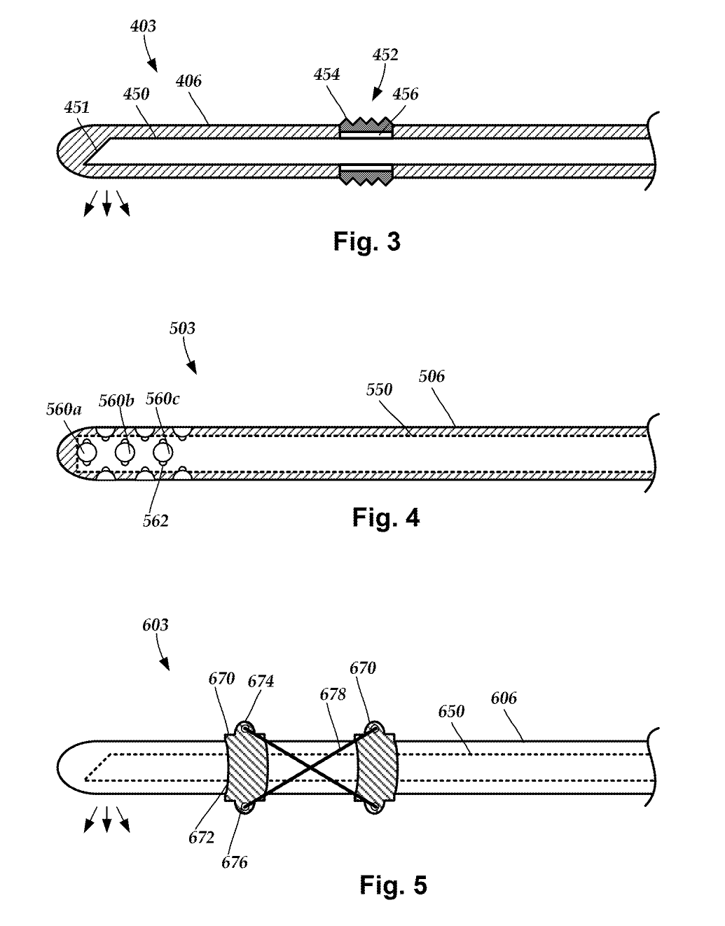

In some embodiments, the control module 102 also includes one or more light sources 111 disposed within the sealed electronics housing 114. The one or more light sources can be, for example, a light emitting diode (LED), laser diode, organic light emitting diode (OLED), or the like. When the control module 102 includes multiple light sources, the light sources can provide light in at a same wavelength or wavelength band or some, or all, of the light sources can provide light at different wavelength or different wavelength bands. When the control module includes one or more light sources 111, the light emitted by the light sources can be directed to an optical fiber (for example, optical fiber 450 in FIG. 3) or other light transmitting body. The optical fiber, or a series of optical fibers, can transmit the light from the one or more light sources 111 through the control module 102 and lead 103 to the light emitter 135 (which can be terminus of the optical fiber). In at least some embodiments, the optical fiber is a single mode optical fiber. In other embodiments, the optical fiber is a multi-mode optical fiber. In some embodiments, the system includes a single optical fiber. In other embodiments, the system may employ multiple optical fibers in series or in parallel.

In other embodiments, the light emitter 135 can also be the light source (a light emitting diode (LED), laser diode, organic light emitting diode (OLED), or the like), or a combination of light sources, with conductors extending along the lead 103 and coupled to the electronic subassembly 110 to provide signals and power for operating the light source. In yet other embodiments, the light source can be disposed elsewhere in the control module 102, on the lead 103, or in another element such as a lead extension or the like.

The stimulation system or components of the stimulation system, including the lead 103 and the control module 102, are typically implanted into the body of a patient. The stimulation system can be used for a variety of applications including, but not limited to brain stimulation, deep brain stimulation, neural stimulation, spinal cord stimulation, muscle stimulation, and the like.

If the lead includes the optional electrodes 134, the electrodes can be formed using any conductive, biocompatible material. Examples of suitable materials include metals, alloys, conductive polymers, conductive carbon, and the like, as well as combinations thereof. In at least some embodiments, one or more of the electrodes 134 are formed from one or more of: platinum, platinum iridium, palladium, palladium rhodium, or titanium. Any suitable number of electrodes 134 can be disposed on the lead including, for example, one, two, three, four, five, six, seven, eight, nine, ten, eleven, twelve, fourteen, sixteen, twenty-four, thirty-two, or more electrodes 134.

The one or more lead bodies 106 are made of a non-conductive, biocompatible material such as, for example, silicone, polyurethane, polyether ether ketone (“PEEK”), epoxy, and the like or combinations thereof. The one or more lead bodies 106 may be formed in the desired shape by any process including, for example, molding (including injection molding), casting, and the like.

One or more terminals (e.g., 310 in FIGS. 2A-2B) are typically disposed along the proximal end of the one or more lead bodies 106 of the stimulation system 100 (as well as any splitters, lead extensions, adaptors, or the like) for electrical connection to corresponding connector contacts (e.g., 314 in FIGS. 2A-2B). The connector contacts are disposed in connectors (e.g., 144 in FIGS. 1-2B; and 322 FIG. 2B) which, in turn, are disposed on, for example, the control module 102 (or a lead extension, a splitter, an adaptor, or the like). Electrically conductive wires, cables, or the like (not shown) extend from the terminals to the light emitter 135 or optional one or more electrodes 134.

The electrically conductive wires (“conductors”) may be embedded in the non-conductive material of the lead body 106 or can be disposed in one or more lumens (not shown) extending along the lead body 106. In some embodiments, there is an individual lumen for each conductor. In other embodiments, two or more conductors extend through a lumen. There may also be one or more lumens (not shown) that open at, or near, the proximal end of the one or more lead bodies 106, for example, for inserting a stylet to facilitate placement of the one or more lead bodies 106 within a body of a patient. Additionally, there may be one or more lumens (not shown) that open at, or near, the distal end of the one or more lead bodies 106, for example, for infusion of drugs or medication into the site of implantation of the one or more lead bodies 106. In at least one embodiment, the one or more lumens are flushed continually, or on a regular basis, with saline, epidural fluid, or the like. In at least some embodiments, the one or more lumens are permanently or removably sealable at the distal end.

FIG. 2A is a schematic side view of one embodiment of a proximal end of one or more elongated devices 300 configured and arranged for coupling to one embodiment of the control module connector 144. The one or more elongated devices may include, for example, one or more of the lead bodies 106 of FIG. 1, one or more intermediate devices (e.g., a splitter, the lead extension 324 of FIG. 2B, an adaptor, or the like or combinations thereof), or a combination thereof.

The control module connector 144 defines at least one port into which a proximal end of the elongated device 300 can be inserted, as shown by directional arrows 312a and 312b. In FIG. 2A (and in other figures), the connector housing 112 is shown having two ports 304a and 304b. The connector housing 112 can define any suitable number of ports including, for example, one, two, three, four, five, six, seven, eight, or more ports.

The control module connector 144 also includes a plurality of connector contacts, such as connector contact 314, disposed within each port 304a and 304b. When the elongated device 300 is inserted into the ports 304a and 304b, the connector contacts 314 can be aligned with a plurality of terminals 310 disposed along the proximal end(s) of the elongated device(s) 300 to electrically couple the control module 102 to the electrodes (134 of FIG. 1) disposed on the paddle body 104 of the lead 103. Each of the terminals 310 can couple to the light emitter 135 or one or more of the optional electrodes 134. Examples of connectors in control modules are found in, for example, U.S. Pat. Nos. 7,244,150 and 8,224,450, which are incorporated by reference.

FIG. 2B is a schematic side view of another embodiment of the stimulation system 100. The stimulation system 100 includes a lead extension 324 that is configured and arranged to couple one or more elongated devices 300 (e.g., one of the lead bodies 106 of FIG. 1, a splitter, an adaptor, another lead extension, or the like or combinations thereof) to the control module 102. In FIG. 2B, the lead extension 324 is shown coupled to a single port 304 defined in the control module connector 144. Additionally, the lead extension 324 is shown configured and arranged to couple to a single elongated device 300. In alternate embodiments, the lead extension 324 is configured and arranged to couple to multiple ports 304 defined in the control module connector 144 (e.g., the ports 304a and 304b of FIG. 1), or to receive multiple elongated devices 300 (e.g., both of the lead bodies 106 of FIG. 1), or both.

A lead extension connector 322 is disposed on the lead extension 324. In FIG. 2B, the lead extension connector 322 is shown disposed at a distal end 326 of the lead extension 324. The lead extension connector 322 includes a connector housing 328. The connector housing 328 defines at least one port 330 into which terminals 310 of the elongated device 300 can be inserted, as shown by directional arrow 338. Each of the terminals 310 can couple to the light emitter 135 or one or more of the optional electrodes 134. The connector housing 328 also includes a plurality of connector contacts, such as connector contact 340. When the elongated device 300 is inserted into the port 330, the connector contacts 340 disposed in the connector housing 328 can be aligned with the terminals 310 of the elongated device 300 to electrically couple the lead extension 324 to the electrodes (134 of FIG. 1) disposed along the lead (103 in FIG. 1).

In at least some embodiments, the proximal end of the lead extension 324 is similarly configured and arranged as a proximal end of the lead 103 (or other elongated device 300). The lead extension 324 may include a plurality of electrically conductive wires (not shown) that electrically couple the connector contacts 340 to a proximal end 348 of the lead extension 324 that is opposite to the distal end 326. In at least some embodiments, the conductive wires disposed in the lead extension 324 can be electrically coupled to a plurality of terminals (not shown) disposed along the proximal end 348 of the lead extension 324. In at least some embodiments, the proximal end 348 of the lead extension 324 is configured and arranged for insertion into a connector disposed in another lead extension (or another intermediate device). In other embodiments (and as shown in FIG. 2B), the proximal end 348 of the lead extension 324 is configured and arranged for insertion into the control module connector 144.

Often the lead passes through or between bones or other rigid tissues of the patient. For example, the distal end of a lead used for spinal cord stimulation will often be positioned within the epidural space of the patient. The portion of the lead transitioning out of the epidural space will be positioned between, or next to, vertebra. As another example, the distal end of the lead used for deep brain stimulation will be positioned within the brain of the patient, but the portion of the lead exiting the skull will often extend through a burr hole in the skull. For optical stimulation leads with an optical fiber, the portion of the lead next to, or between, bones or other rigid tissues can be pinched or otherwise worn or compressed by the bone or rigid tissue which may damage the optical fiber. As described herein, a portion of the lead can be made to produce a cushion at these positions along the lead to reduce or prevent the damage to the optical fiber.

FIG. 3 illustrates a distal end of an optical lead 403 with an optical fiber 450 extending along the lead and a lead body 406 disposed around the lead. At one or more positions along the lead 403, a flex zone 452 can be formed using an elastomeric structure 454 spaced apart from the lead by a cushion region 456. In other embodiments, there is no cushion region and the elastomeric structure 454 is disposed next to the optical fiber 450.

The optical fiber 450 can be any suitable optical fiber including any single-mode or multi-mode optical fiber. In at least some embodiments, the optical fiber 450 may have a reflective surface 452 to reflective light out of a side of the lead, as illustrated in FIG. 3. In other embodiments, the optical fiber may emit light out of the distal end of the lead 403. Any other suitable arrangement for light emission, such as in a ring around the lead or at multiple points around the lead, can be used. In addition, in at least some embodiments, the lead 403 can include multiple optical fibers.

The lead body 406 can be simply the cladding of the optical fiber 450 or can include other layers in addition to, or as an alternative to, the cladding. The cladding of the optical fiber can be made of any suitable material such as, for example, polyvinyl chloride, polyurethane, or the like. In addition, materials such as, for example, silicone, polyurethane, polyether ether ketone, to the like may be disposed around the cladding or optical fiber 450 to form, at least in part, the lead body 406.

The elastomeric structure 454 of the flex zone 452 can be made of any elastomeric material such as, for example, any rubber material, silicone, or the like. In at least some embodiments, the elastomeric structure 454 can have features on the exterior surface, such as, for example, pyramidal features, bumps, hemispherical features, ridged features, or the like, to provide further cushioning to the optical fiber 450 in the lead 403. In at least some embodiments, such features also (or alternatively) may be present on the interior surface of the elastomeric structure.

In at least some embodiments, the elastomeric structure 454 can extend at least 2, 4, 5, 10, 15, 20, 25, or more millimeters along the longitudinal length of the lead. In at least some embodiments, the elastomeric structure 454 can be disposed around the entire circumference of the lead, although in other embodiments, the elastomeric structure may be disposed around less than the full circumference (for example, 75, 50, or 25 percent of the circumference) or may divided into multiple structures arranged around the circumference of the lead. In at least some embodiments, the one or more flex zones are provided at one or more regions of the lead that are expected to be near bone or rigid tissue of a typical patient or typical range of patients.

The cushion region 456 can be filled with air or other gas or liquid (e.g., saline or water). The elastomeric structure 454 and optional cushion region 456 are provided to protect or resist damage to the optical fiber 450. In at least some embodiments, the cushion region 456 has a radial width of at least 0.02, 0.03, 0.05, 0.1, 0.2, 0.4, 0.5, 1, or more millimeters. In at least some embodiments, the cushion region 456 has the same longitudinal length as the elastomeric structure 454, although in other embodiments the longitudinal length of the cushion region 456 can be greater or less than that of the elastomeric structure 454. In at least some embodiments, the cushion region 456 can be disposed around the entire circumference of the lead, although in other embodiments, the cushion region 456 may be disposed around less than the full circumference (for example, 75, 50, or 25 percent of the circumference) or may divided into multiple regions arranged around the circumference of the lead. In at least some other embodiments, the lead includes the elastomeric structure without a corresponding cushion region.

In at least some embodiments, it can be desirable to selectively emit light of different colors or wavelengths. FIG. 4 illustrate one embodiment of a lead 503 with an optical fiber 550 and an array of filters 560a, 560b, 560c through which light is emitted. The filters 560a, 560b, 560c can be disposed on the exterior of the lead body 506, the interior of the lead body, or embedded in the lead body, or any combination thereof. In at least some embodiments, the different filters 560a, 560b, 560c can allow the emission of light having different colors, wavelengths, or wavelength bands. For example, filters 560a may allow the emission of red light, filters 560b may allow the emission of near infrared light, and filters 560c may allow the emission of yellow light. In at least some embodiments, the wavelengths or wavelength bands can be in a range of 500 to 1000 nm or in a range of 600 to 900 nm.

In at least some embodiments, the filters 560a, 560b, 560c can be passive components that simply filter the light provided by the optical fiber 550. In at least some others embodiments, the filters 560a, 560b, 560c can be active components and may include contacts 562 that are coupled electrically to the control module or other control device to turn the filters on and off. In the on position, the filter allows light to pass through the filter and, in the off position, the filter resists or prevents the passage of light through the filter. As an example, the filters 560a, 560b, 560c can be similar to those used in LCD displays and the like. Examples of such filters 560a, 560b, 560c can include a layer of liquid crystal and a color filter layer, although other types of filters are also known and can be used. In at least some embodiments, the color, wavelength, or wavelength band of filter 560a, 560b, 560c may also be selected by signals send to the filter through the contacts 562.

The optical fiber 550 can be any suitable optical fiber and, in at least some embodiments, can be a multi-mode optical fiber. In at least some embodiments, the control module can provide light of the desired stimulation color(s), wavelength(s), or wavelength band(s) to the optical fiber 550 and that light will be emitted through the corresponding filters 560a, 560b, 560c. The stimulation can be color/wavelength/band modulated by controlling the light provided to the optical fiber 550. In other embodiments, light with multiple colors, wavelengths, or wavelength bands can be provided to the optical fiber 550 and the emission of light can be modulated by selecting which of the filter 560a, 560b, 560c are turned on. This latter arrangement can also be useful to directional modulate the stimulation light as filters 560a, 560b, 560c can be turned on in the desired direction(s).

As an alternative to an optical fiber 550, one or more light emitting diodes (LEDs), organic light emitting diodes (OLEDs), laser diodes, or other light sources may be disposed at the distal end of the lead to provide the light that passes through the filters 560a, 560b, 560c. For example, one or more white light sources can be disposed at the distal end of the lead. Alternatively, one or more light sources for each of multiple colors, wavelengths, or wavelength bands can be disposed at the distal end of the lead. These light sources can be electrically coupled to the control module by conductors that extend along the lead. The control module can then direct turning on and off the light sources, as well as other parameters such as light intensity, pulse frequency, pulse width, and the like using signals sent to the light source(s) over the conductors.

To facilitate maintaining the position (for example, longitudinal or lateral position) of an optical lead in the patient tissue, one or more stabilizers can be provided on, or with, the lead. In addition, for leads where the light is emitted from the side of the lead, the stabilizer can prevent or resist rotation. FIG. 5 illustrates one embodiment of a lead 603 having an optical fiber 650 and a lead body 603 with one or more lead stabilizers 670. In the illustrated embodiment, each lead stabilizer 670 includes a body 672 with one or more tabs 674 extending from the body and one or more suture holes 676 in the tabs. A suture 678 can fasten the lead stabilizer 670 and lead 603 to patient tissue by passing through the suture tab. In the illustrated embodiment, the suture 678 also passes through one or more suture holes 676 of each of two lead stabilizers 670 in a “criss-cross” pattern. Other suture patterns can also be used.

In at least some embodiments, the one or more stabilizers 670 can be molded or formed with the lead body 606. In other embodiments, the one or more stabilizers 670 can be overmolded the lead body 606. In yet other embodiments, the one or more stabilizers 670 can be slid onto the lead body 606 or wrapped around the lead body 606 or adhesively attached to the lead body to affix stabilizer to the lead body. The one or more stabilizers 670 may form a compression or frictional fit with the lead body 606 to prevent or reduce longitudinal or rotational movement of the lead body relative to the stabilizer.

It will be understood that an optical stimulation lead can include one or more flex zones, one or more filters, one or more stabilizers, or any combination thereof.

Although the leads in FIGS. 3-5 are illustrated as emitting light from a side of the lead (e.g., side emission), it will be understood that in other embodiments, the light can be emitted from the distal end of the lead (i.e., forward emission) or in a ring around the circumference of the lead (i.e., circumferential emission), or any combination of forward, side, or circumferential emission.

Although the leads in FIGS. 3-5 are illustrated using an optical fiber for emission of lead, it will be understood that in other embodiments, light can be emitted using one or more light sources at the distal portion of the lead. Examples of suitable light sources include, but are not limited to, LEDs, OLEDs, laser diodes, and the like.

The optical leads described herein can be used for providing spinal cord stimulation, deep brain stimulation, peripheral nerve stimulation, or any other type of stimulation of patient tissue. In at least some embodiments, the optical leads can be implanted using a cannula or needle.

Although the leads in FIGS. 3-5 are illustrated as only providing optical stimulation, it will be understood that one or more electrodes can be disposed on the lead to provide electrical stimulation. Examples of electrical stimulation leads that can be modified to also provide optical stimulation, as described herein, are provided in the references cited above.

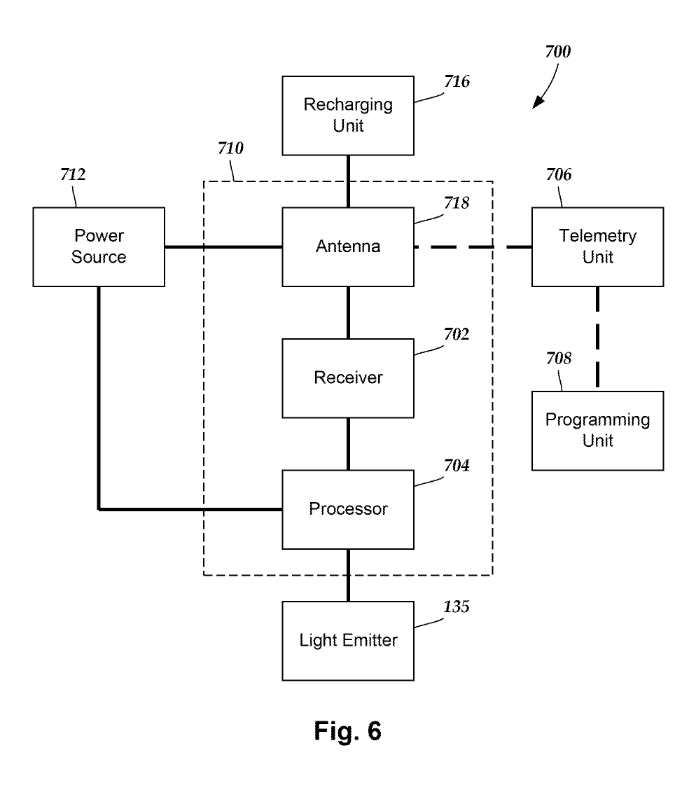

FIG. 6 is a schematic overview of one embodiment of components of an optical stimulation system 700 including an electronic subassembly 710 disposed within a control module. It will be understood that the optical stimulation system can include more, fewer, or different components and can have a variety of different configurations including those configurations disclosed in the stimulator references cited herein.

Some of the components (for example, a power source 712, an antenna 718, a receiver 702, and a processor 704) of the optical stimulation system can be positioned on one or more circuit boards or similar carriers within a sealed housing of an implantable pulse generator, if desired. Any power source 712 can be used including, for example, a battery such as a primary battery or a rechargeable battery. Examples of other power sources include super capacitors, nuclear or atomic batteries, mechanical resonators, infrared collectors, thermally-powered energy sources, flexural powered energy sources, bioenergy power sources, fuel cells, bioelectric cells, osmotic pressure pumps, and the like including the power sources described in U.S. Pat. No. 7,437,193, incorporated herein by reference.

As another alternative, power can be supplied by an external power source through inductive coupling via the optional antenna 718 or a secondary antenna. The external power source can be in a device that is mounted on the skin of the user or in a unit that is provided near the user on a permanent or periodic basis.

If the power source 712 is a rechargeable battery, the battery may be recharged using the optional antenna 718, if desired. Power can be provided to the battery for recharging by inductively coupling the battery through the antenna to a recharging unit 716 external to the user. Examples of such arrangements can be found in the references identified above.

In one embodiment, light is emitted by the light emitter 135 of the lead body to stimulate nerve fibers, muscle fibers, or other body tissues near the optical stimulation system. The processor 704 is generally included to control the timing and other characteristics of the optical stimulation system. For example, the processor 704 can, if desired, control one or more of the timing, pulse frequency, strength, duration, and waveform of the optical stimulation. In addition, the processor 704 can select one or more of the optional electrodes to provide electrical stimulation, if desired. In some embodiments, the processor 704 selects which of the optional electrode(s) are cathodes and which electrode(s) are anodes.

Any processor can be used and can be as simple as an electronic device that, for example, produces optical stimulation at a regular interval or the processor can be capable of receiving and interpreting instructions from an external programming unit 708 that, for example, allows modification of stimulation characteristics. In the illustrated embodiment, the processor 704 is coupled to a receiver 702 which, in turn, is coupled to the optional antenna 718. This allows the processor 704 to receive instructions from an external source to, for example, direct the stimulation characteristics and the selection of electrodes, if desired.

In one embodiment, the antenna 718 is capable of receiving signals (e.g., RF signals) from an external telemetry unit 706 which is programmed by the programming unit 708. The programming unit 708 can be external to, or part of, the telemetry unit 706. The telemetry unit 706 can be a device that is worn on the skin of the user or can be carried by the user and can have a form similar to a pager, cellular phone, or remote control, if desired. As another alternative, the telemetry unit 706 may not be worn or carried by the user but may only be available at a home station or at a clinician's office. The programming unit 708 can be any unit that can provide information to the telemetry unit 706 for transmission to the optical stimulation system 700. The programming unit 708 can be part of the telemetry unit 706 or can provide signals or information to the telemetry unit 706 via a wireless or wired connection. One example of a suitable programming unit is a computer operated by the user or clinician to send signals to the telemetry unit 706.

The signals sent to the processor 704 via the antenna 718 and the receiver 702 can be used to modify or otherwise direct the operation of the optical stimulation system. For example, the signals may be used to modify the stimulation characteristics of the optical stimulation system such as modifying one or more of stimulation duration, pulse frequency, waveform, and stimulation amplitude. The signals may also direct the optical stimulation system 700 to cease operation, to start operation, to start charging the battery, or to stop charging the battery. In other embodiments, the stimulation system does not include the antenna 718 or receiver 702 and the processor 704 operates as programmed.

Optionally, the optical stimulation system 700 may include a transmitter (not shown) coupled to the processor 704 and the antenna 718 for transmitting signals back to the telemetry unit 706 or another unit capable of receiving the signals. For example, the optical stimulation system 700 may transmit signals indicating whether the optical stimulation system 700 is operating properly or not or indicating when the battery needs to be charged or the level of charge remaining in the battery. The processor 704 may also be capable of transmitting information about the stimulation characteristics so that a user or clinician can determine or verify the characteristics.

The above specification provides a description of the structure, manufacture, and use of the invention. Since many embodiments of the invention can be made without departing from the spirit and scope of the invention, the invention also resides in the claims hereinafter appended.

What is claimed as new and desired to be protected by Letters Patent of the United States is:

1. An optical stimulation lead, comprising:
a lead body comprising a distal portion and a proximal portion;
an optical fiber disposed within the lead body and configured and arranged to emit light from a distal portion of the optical fiber; and
at least one flex zone disposed along the distal portion of the lead body, wherein each flex zone comprises an elastomeric structure that is made of a flexible material different from the lead body.

2. The optical stimulation lead of claim 1, wherein the elastomeric structure comprises a rubber material.

3. The optical stimulation lead of claim 1, wherein the lead body comprises a cladding of the optical fiber.

4. The optical stimulation lead of claim 1, wherein the elastomeric structure comprises a plurality of features on an exterior surface of the elastomeric structure.

5. The optical stimulation lead of claim 4, wherein the features are pyramidal features, bumps, hemispherical features, ridged features, or any combination thereof on the exterior surface.

6. The optical stimulation lead of claim 1, further comprising a cushion region between the elastomeric structure and the optical fiber, wherein the cushion region has a radial width of at least 0.03 millimeters.

7. The optical stimulation lead of claim 1, wherein the elastomeric structure extends around an entire circumference of the optical fiber.

8. An optical stimulation system, comprising:
the optical stimulation lead of claim 1; and
a control module coupleable to the optical stimulation lead, the control module comprising
a housing, and
a light source disposed in the housing and configured and arranged to provide light to the optical fiber of the optical stimulation lead.

9. An optical stimulation lead, comprising:
a lead body comprising a distal portion and a proximal portion;
at least one light emitter disposed within the lead body and configured and arranged to emit light from a distal portion of the lead body; and
a plurality of light filters disposed along the distal end of the lead and configured and arranged to receive the emitted light from the at least one light emitter and permit passage of light through the light filter of a predetermined wavelength or range of wavelengths, wherein at least two of the light filters permit passage of light for different wavelengths or different range of wavelengths from each other.

10. The optical stimulation lead of claim 9, wherein the at least one light emitter comprises at least one optical fiber.

11. The optical stimulation lead of claim 10, wherein the at least one optical fiber comprises at least one multimode optical fiber.

12. The optical stimulation lead of claim 9, wherein the at least one light emitter comprises at least one light source selected from the group consisting of a light emitting diode, an organic light emitting diode, a laser diode, or any combination thereof.

13. The optical stimulation lead of claim 12, wherein the at least one light source is a plurality of light sources, wherein at least two of the light sources produce light of different wavelengths from each other.

14. The optical stimulation lead of claim 9, wherein the light filters are configured and arranged to be turned on or off by received electrical signals.

15. An optical stimulation system, comprising:
the optical stimulation lead of claim 9; and
a control module coupleable to the optical stimulation lead, the control module comprising
a housing, and
an electronic subassembly disposed in the housing and configured and arranged to provide electrical signals or light to the at least one light emitter of the optical stimulation lead.

16. An optical stimulation lead, comprising:
a lead body comprising a distal portion and a proximal portion;
at least one light emitter disposed within the lead body and configured and arranged to emit light from a distal portion of the lead body; and
a plurality of stabilizers disposed along the distal portion of the lead body, each stabilizer comprising a body disposed around the lead body, at least one tab extending from the body, and at least one suture hole formed in the at least one tab.

17. The optical stimulation lead of claim 16, wherein the at least one stabilizer is molded with the lead body to form a unitary arrangement of the lead body and the at least one stabilizer.

18. The optical stimulation lead of claim 16, wherein the at least one stabilizer is affixed to the lead body by overmolding the at least one stabilizer over the lead body.

19. The optical stimulation lead of claim 16, wherein the at least one stabilizer is slideably disposed on the lead body.

20. An optical stimulation system, comprising:
the optical stimulation lead of claim 16; and
a control module coupleable to the optical stimulation lead, the control module comprising
a housing, and
an electronic subassembly disposed in the housing and configured and arranged to provide electrical signals or light to the at least one light emitter of the optical stimulation lead.
